(19) United States
(12) Patent Application Publication
(10) Pub. No.: US 2016/0000875 A1
(43) Pub. Date: Jan. 7, 2016

(54) GM-CSF FOR TREATMENT OF CHRONIC ORAL MUCOSITIS

(71) Applicant: Reponex Pharmaceuticals ApS, Hørsholm (DK)

(72) Inventor: Lars Heslet, Gentofte (DK)

(73) Assignee: Reponex Pharmaceuticals ApS, Hørsholm (DK)

(21) Appl. No.: 14/769,424

(22) PCT Filed: Feb. 19, 2014
(86) PCT No.: PCT/EP2014/053252

§ 371 Date: Aug. 20, 2015

(30) Foreign Application Priority Data
Feb. 20, 2013 (EP) 13155957.7

(51) Int. Cl. A61K 38/19

(52) U.S. Cl. CPC A61K 38/193

(57) ABSTRACT

The present invention relates to compositions comprising granulocyte-macrophage colony-stimulating factor (GM-CSF), or homologues thereof, and its use for treatment, prevention or alleviation of oral mucositis. The composition is preferably an aqueous solution forlocaloral administration.

### FIELD OF INVENTION

The present invention relates to compositions comprising granulocyte-macrophage colony-stimulating factor (GM-CSF), or homologues thereof, and its use for treatment, prevention or alleviation of post-therapeutic oral mucositis.

### BACKGROUND OF INVENTION

Mucositis occurs when cancer treatments break down the rapidly divided epithelial cells lining the GI tract, particularly in the oral cavity, leaving the mucosal tissue open to ulceration and infection. Oral Mucositis is probably the most common, debilitating complication of cancer surgery, chemotherapy and radiation. It occurs in 20-40% of patients treated with chemotherapy alone and up to 50% of patients receiving combination radiation and chemotherapy, especially those with head and neck cancer. Drugs such as doxorubicin, paclitaxel, and capecitabine are commonly used in breast cancer and frequently associated with oral mucositis. The consequences of mucositis can be mild requiring little intervention to severe (hypovolemia, electrolyte abnormalities, and malnutrition) that may result in fatal complications.

Primary acute oral mucositis is the acute inflammatory and ulcerative reaction of the oral mucosa, a complication often accompanying radiation therapy to the head and neck region and chemotherapy. It is usually transient in nature but it also represents an important clinical problem as it is a painful, debilitating, dose-dependent side effect for which there is no widely acceptable prophylaxis or effective treatment. Clinical manifestations progress from erythema, cracking, and inflammation, to pain, bleeding and ulceration.

The primary acute stage of the oral mucositis appears subsequent to irradiation and/or chemotherapy, and continues while treatment is ongoing. When treatment ceases also the insult to the mucosa ceases, and the inflammatory state of the oral mucosal lining will disappear. Some mucous membrane atrophy remains in the symptom-free stage following an acute mucositis. However, for a subset of patients in the symptom-free stage after the primary acute oral mucositis they will progress into a chronic stage, a stage where the patient no longer receives chemotherapy and/or radiotherapy. The oral mucositis thus developed is post-therapeutic and/or secondary, and as such not directly or immediately linked to and caused by treatment.

Oral mucositis is generally not well-managed and prophylactic at best providing palliative relief from the accompanying pain and management of concurrent infections. Clinicians and nursing staff currently rely on the protocol used in their centers or their individual experiences to make the best out of these agents.

GM-CSF has been tested for management of oral mucositis in the primary acute phase. A range of smaller studies show different results, while a larger double-blind randomized placebo-controlled study show that prophylactic use of mouthwashes does not reduce the severity and frequency of chemotherapy-induced oral mucositis (Dazzi et al. Annals of oncology 14:559-563, 2003). The recommendation of the US National Cancer Institute however remains that granulocyte-macrophage colony-stimulating factor mouthwashes should be avoided for oral mucositis.

### SUMMARY OF INVENTION

Provided herein is a composition comprising granulocyte-macrophage colony-stimulating factor (GM-CSF), or a functional variant or homologue thereof, for use in the treatment, prevention or alleviation of post-therapeutic oral mucositis.

In some embodiments, the compositions of the invention comprises GM-CSF, hyaluronic acid, and/or sucralfate, and/or algenate

Oral mucositis according to the present invention is mucositis in the oral cavity, and/or the throat, and/or the esophagus.

In one embodiment said post-therapeutic mucositis is subsequent to an acute mucositis caused by chemotherapy and/or radiotherapy, wherein said acute mucositis caused by chemotherapy/or radiotherapy has subsided partially or completely.

Further, said post-therapeutic mucositis is subsequent to a symptom-free stage characterized by atrophy of the oral mucosal lining, said symptom-free stage following an acute mucositis caused by chemotherapy and/or radiotherapy.

Said post-therapeutic oral mucositis may be associated with mucous membrane atrophy of the oral cavity, and said atrophic mucous membrane of the oral cavity may be regenerated by the present treatment.

In an embodiment, the composition for use according to the present invention is to be administered prior to and/or during and/or after chemotherapy and/or radiotherapy.

In one preferred embodiment, the composition for use comprising GM-CSF, or a functional variant or homologue thereof, is an aqueous solution, wherein said aqueous solution is preferably administered orally or locally to the oral cavity. Said aqueous solution may be a mouth wash.

In one embodiment, the aqueous solution according to the present invention further comprises an adhesive or a gelling agent or a thickener, such as an alginate, hyaluronic acid or sucralfate.

Alginate is well known as an additive which by absorption of water can form a gel which can function as an adhesive in the formulations of the present invention. In some embodiments the alginate used in formulations of the invention is any one of sodium alginate, potassium alginate or calcium alginate.

Alginate may be present in the formulations of the present invention in an amount of between 5 and 500 mg/ml, such as between 5 and 250 mg/ml such as between 5 and 125 mg/ml. In one embodiment, alginate is present in about 50 mg/ml in the formulations of the present invention.

In one embodiment, the aqueous solution according to the present invention further comprises hyaluronic acid, which is well known to function as a thickener which also play a role in postoperative wound healing.

In one embodiment, the aqueous solution according to the present invention further comprises sucralfate (except for in patients suffering from chronic renal failure). Sucralfate may be administered in a dosage ranging from 0.01 gram to 5 grams/dose. Sucralfate is a cytoprotective agent, an oral gastrointestinal medication primarily indicated for the treatment of active duodenal ulcers.

In one embodiment, the aqueous solution according to the present invention further comprises the thickener Xanthan gum. Xantan gum is a polysaccharide secreted by the bacterium Xanthomonas campestris, used as a food additive and rheology modifier, commonly used as a food thickening agent (in salad dressings, for example) and a stabilizer.

Typical gelling agents which may be used in the solutions of the present invention include natural gums, starches, pectins, agar-agar and gelatin. Often they are based on polysaccharides or proteins.

Examples are:

Alginic acid (E400), sodium alginate (E401), potassium alginate (E402), ammonium alginate (E403), calcium alginate (E404)-polysaccharides from brown algae

Agar (E406, a polysaccharide obtained from red algaes)

Carrageenan (E407, a polysaccharide obtained from red seaweeds)

Locust bean gum (E410, a natural gum polysaccharide from the seeds of the Carob tree)

Pectin (E440, a polysaccharide obtained from apple or citrus-fruit)

Gelatin (E441, made by partial hydrolysis of animal collagen)

These gelling agents may be used in similar concentrations as alginic acid for thickening the solutions of the present invention.

In one embodiment, the compositions of the invention further comprises a mixture of Polyvinylpyrrolidone (PVP), Hyaluronic acid (sodium hyaluronate) and glycyrrhetinic acid as an adhesive (this composition is known as Gelclair). In one embodiment, the compositions of the invention further comprises a mixture of Polyvinylpyrrolidone (PVP), and Hyaluronic acid (sodium hyaluronate) as an adhesive.

In one embodiment, the composition comprising GM-CSF, or a functional variant or homologue thereof, is to be administered at an effective amount, such as from between 100 to 1000 microgram per dose, for example 200-800 microgram per dose, such as at about 300 microgram per dose. Said dose may be administered between two and five times daily, for 7 days to several months

The composition for use according to the present invention is preferably contained in the oral cavity by agitating or swirling of the solution within the oral cavity for a period of from 1 minute to 60 minutes.

### DETAILED DESCRIPTION OF THE INVENTION

Provided herein is a composition comprising granulocyte-macrophage colony-stimulating factor (GM-CSF), or functional variant or homologue thereof, for use in the treatment, prevention or alleviation of post-therapeutic oral mucositis.

Said post-therapeutic or secondary oral mucositis is developed subsequent to a primary acute oral mucositis, after the primary acute oral mucositis has subsided and developed into a symptom-free stage characterized by an atrophic oral mucosal lining.

The post-therapeutic or secondary oral mucositis is thus not a direct consequence of, or directly triggered by, therapy, such as chemotherapy or radiotherapy

Also provided herein is a composition comprising granulocyte-macrophage colony-stimulating factor (GM-CSF), or functional variant or homologue thereof, for use in the treatment, prevention or alleviation of mucous membrane atrophy of the oral cavity, wherein said atrophy is associated with inflammation and/or infection. It follows that the composition may be used for regenerating an atrophic mucous membrane of the oral cavity, wherein said atrophic mucous membrane is subsequent to an acute oral mucositis.

The oral cavity may be defined herein as the mouth or mouth cavity, being bounded laterally and in front by the alveolar process (containing the teeth), posteriorily by the isthmus of the fauces, superiorly or the roof is formed by hard and soft palate and inferiorly or the floor of the mouth is formed by the mylohyoid muscles and is occupied mainly by the tongue. For the purposes of the present invention, oral cavity refers to the uppermost part of the gastrointestinal tract including the oral cavity and upper part of the esophagus, being separated by and including the epiglottis.

The oral mucosa is the mucous membrane epithelium lining the inside of the mouth or oral cavity including the epiglottis. The epiglottis is a flap that is made of elastic cartilage tissue covered with a mucous membrane, attached to the entrance of the larynx. It projects obliquely upwards behind the tongue and the hyoid bone, pointing dorsally. The epiglottis guards the entrance of the glottis, the opening between the vocal folds. It prevents food from going into the trachea and instead directs it to the esophagus, which is posterior.

### Oral Mucositis

Mucositis is the painful inflammation and ulceration of the mucous membranes lining the digestive tract, usually as an adverse effect of chemotherapy and radiotherapy treatment for cancer.

Mucositis can occur anywhere along the gastrointestinal (GI) tract, but oral mucositis refers to the particular inflammation and ulceration that occurs in the mouth, and/or the throat, and/or the esophagus. Oral mucositis affects almost all patients undergoing high-dose chemotherapy and hematopoietic stem cell transplantation (HSCT), 80% of patients with malignancies of the head and neck receiving radiotherapy, and a wide range of patients receiving chemotherapy. For most cancer treatment, about 5-15% of patients get mucositis. However, with 5-fluorouracil (5-FU), up to 40% get mucositis, and 10-15% get grade 3-4 oral mucositis. 75-85% of bone marrow transplantation recipients experience oral mucositis.

Signs and symptoms of mucositis include: Red, shiny, or swollen mouth and gums, Blood in the mouth, Sores in the mouth or on the gums or tongue. Soreness or pain in the mouth or throat, severely aggravated by tooth brushing. Difficulty swallowing or talking, Feeling of dryness, mild to severe burning, or pain when eating food a process most frequently accompanied by coughing Soft, whitish patches or pus in the mouth or on the tongue which may be a sign of Candida infection, a sign of reduced local host defense. Increased mucus formation in the mouth. The taste is often altered.

Oral mucositis can be severely painful. The degree of pain is usually related to the extent of the tissue damage, and may cause the patient to experience trouble speaking, eating, or even opening the mouth (please refer to table 1)

TABLE 1
Oral Mucositis grading scale (after WHO standard)
Signs and symptoms

| | Grade 0 | Grade 1 | Grade 2 | Grade 3 | Grade 4 |
|---|---|---|---|---|---|
| Overall | No | Mild | Moderate | | Severe mucositis |
| evaluation | mucositis | mucositis | mucositis | | |
| Erythema | None | Erythema | Ulcers + | Extensive | Extensive |
| & ulcer | | | Erythema | Erythema | Erythema |
| | | | no ulcers | Ulcers | Ulcers Bleeding |
| | | | | | mucosa |
| Peroral | Normal | Mild pain | Swallowing | Cannot | Oral feeding |
| food intake | | | solid food | swallow solid | impossible |
| | | | with | food | |
| Body | Normal | Normal | Moderately | Difficulties in | Cannot maintain |
| weight | stable | stable BWT | reduced | maintaining | BWT on peroral |
| (BWT) | BWT | | BWT | BWT | diet |
| Pain | No pain | Mild pain^{a} | Moderate^{b} | Severe^{c} when | Severe |
| | | soreness | Only when | ingesting food | constant^{d} |
| | | | ingesting | Moderate | |
| | | | food | without food | |
| | | | | intake | |
| Host | No positive | + Oral D + R | ++ Oral D + R | +++ Oral | +++ Oral D + R |
| Defense^{w} | Oral D + R | No oral AB | + Oral AB | D + R⁽ⁱ⁾ | ++ Oral AB |
| | No oral AB | − Systemic | − Systemic | ++ Oral AB | + Systemic AB, |
| | or systemic | AB | AB | − Systemic AB | often related to |
| | AB | | | | sepsis |

^{w}As documented by local and systemic infection − = Never; + = Sometimes; ++ = Frequent; +++ = Constant: D + R = surveillance culture for Candida spp
Adapted from WHO oral/toxicity scale

In grade 3 oral mucositis, the patient is unable to eat solid food, and in grade 4, the patient is unable to consume liquids as well. Radiotherapy to the head and neck is associated with Grade 3 and Grade 4 oral mucositis, often exceeding 50% of patients. Among patients undergoing head and neck radiotherapy, pain and decreased oral function may persist long after the conclusion of therapy. Fractionated radiation dosage increases the risk of mucositis to >70% of patients in most trials.

Although these agents are all known to cause mucositis, there is still a great deal of patient variation in the severity of mucositis, even among patients receiving identical treatment regimens. Although it is not clear to what extent patient factors correlate with mucositis prevalence, several are identified in the literature, including age, nutritional status, and oral health. Other risk factors include: radiation treatment history; salivary gland dysfunction; physical, chemical and thermal mucosal injury; microbial flora; and graft-versus host disease.

Cancer patients undergoing chemotherapy usually become symptomatic four to five days after beginning treatment, reaching a peak at around day 10, and then slowly improving over the course of a few weeks. Mucositis associated with radiotherapy usually appears at the end of the second week of treatment and may last for six to eight weeks. This constitutes the primary acute oral mucositis.

The pathophysiology of mucositis can be divided into its 5 stages; including an initiation phase, a message generation phase, a signaling and amplification phase, an ulceration phase, and a healing phase. Different cytokines are responsible for the various stages. The initiation phase is caused by the production of free radicals caused by the chemo- or radio-therapy, which damages cell DNA. This causes the production of cell transcription factors such as NF-κB, which upregulates inflammatory cytokines, marking the beginning of the ulceration phase.

The epithelial cells of the oral mucosa undergo rapid turnover, usually every 7 to 14 days, which makes these cells susceptible to the effects of cytotoxic therapy. Both chemotherapy and radiation therapy can interfere with the maturity and cellular growth of epithelial cells, causing changes to normal turnover and cell death.

As a result of cell death in reaction to chemo- or radio-therapy, the mucosal lining of the mouth becomes thin or atrophic, may slough off and then become red, inflamed and ulcerated. The ulcers may become covered by a yellowish white fibrin clot called a pseudomembrane. Peripheral erythema is usually present. Ulcers may range from 0.5 cm to greater than 4 cm.

Patients having been treated with chemotherapy and/or radiotherapy experience an atrophy of the oral cavity, which atrophy will persist to become a chronic condition. This atrophy will in itself be disabling—although considerably less than when the acute mucositis is ongoing—but will also predispose the oral cavity for further inflammation and infections at a later stage (post-therapeutic mucositis).

In one embodiment, a post treatment chronic oral mucositis is defined as mucositis in the oral cavity or in the oesophagus, wherein the mucositis has not entered the healing phase and is not healed, or has recurred 1 week after treatment has stopped or paused.

In one embodiment, a post treatment chronic oral mucositis is defined as mucositis in the oral cavity or in the oesophagus, wherein the mucositis has not entered the healing phase, and is not healed or has recurred at any one of the following time points 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, such as 56 weeks after treatment has stopped or paused.

In one embodiment, the compositions of the invention are made for prophylactic, preemptive treatment or for the treatment of atrophy of the mucosal lining of the oral cavity.

In one embodiment, the compositions of the invention are made for prophylactic, preemptive treatment or for the treatment of atrophy of the mucosal lining of the oral cavity in connection with radiation and/or chemotherapy.

In one embodiment, the compositions of the invention are made for prophylactic, preemptive treatment or for the treatment of atrophy of the mucosal lining of the oral cavity in connection with radiation and/or chemotherapy.

In one embodiment, the compositions of the invention are made for prophylactic, preemptive treatment or for the treatment of inflammation of the mucosa in the oral cavity.

In one embodiment, the compositions of the invention are made for prophylactic, preemptive treatment or for the treatment of ulcers in the mucosa in the oral cavity. In one embodiment, the compositions of the invention are made for prophylactic, preemptive treatment or for the treatment of atrophy of the mucosal lining of the oral cavity, wherein the atrophy is at least partly caused by poor oral or dental health, smoking or chewing tobacco, drinking alcohol, gender (females appear to be more likely than males to develop mucositis), dehydration, low body mass index, kidney disease, diabetes or HIV/AIDS, previous cancer treatment, chronic irritation from ill-fitting prostheses or faulty restorations (can predispose patients to the development of oral mucositis due to local irritation), and trauma, patients with hematologic malignancies (have an increased rate of oral mucositis compared with those with solid tumors), hyposalivation prior to and during treatment (is associated with an increased risk of oral mucositis), and the use of methotrexate for chronic GVHD prophylaxis (may exacerbate lesions of oral mucositis).

In one embodiment, the compositions of the invention are made for prophylactic, preemptive treatment or for the treatment of atrophy of the mucosal lining of the oral cavity in connection with neutropenia.

Oral mucositis occurs independently of oral mucosal infections of viral and fungal etiology, but it may be exacerbated by such concomitant infections.

Patients with oral mucositis and neutropenia (a type of white blood cell deficiency) have a relative risk of septicemia (a systemic, toxic illness caused by the invasion of the bloodstream by virulent bacteria coming from a local infection) more than 4 times that of patients with neutropenia only.

### Treatment of Mucositis

Up until today, oral mucositis is generally not well-managed, as no evidence based cure is available. Thus, treatment is mainly symptomatic to cause relief from pain. Oral mucositis may be minimized and partly managed by good oral hygiene, avoidance of spicy, acidic, hard, and hot foods and beverages, use of mild-flavored toothpastes, mouthwashes (saline-peroxide and Mugard mouth rinse), ice chips/cryotherapy, pain relief (benzocaine, dyclonine, lidocaine, oral opioid medications), treatment of infections (antibiotics) and/or antacids.

A clinical trial has been conducted aiming to assess the effect of a GM-CSF mouthwash to prevent and treat radiation-induced acute mucositis (clinicaltrials.gov identifier NCT00293462). Dazzi et al. (Annals of oncology 14:559-563, 2003) has previously reported that prophylactic mouthwashes with GM-CSF do not reduce the severity and frequency of chemotherapy-induced oral mucositis in a double-blind randomized placebo-controlled study. It remains unclear from the literature if GM-CSF has an effect on primary acute mucositis but the recommendation of the US National cancer Institute remains that no GM-CSF mouthwashes should be used for acute oral mucositis (“Specific recommendations against specific practices include the following: •No systemic glutamine for the prevention of gastrointestinal mucositis. •No sucralfate or antibiotic lozenges for radiation-induced mucositis. •No granulocyte-macrophage colony-stimulating factor mouthwashes” citation from the National Cancer Institute web page 6 Feb. 2014: http://www.cancer.gov/cancertopics/pdq/supportivecare/oralcomplications/Health Professional/page5)).

The present inventor have now shown that GM-CSF administered to a patient with oral mucositis developed post-therapeutically i.e. after treatment with chemotherapy effectively treated the post-therapeutic oral mucositis and reverted the atrophy of the mucosal lining of the oral cavity. Treatment may be curative, ameliorating or prophylactic. Preferably, the patient is treated and the composition comprising GM-CSF is administered before and/or during and/or after an acute attack of post-therapeutic oral mucositis.

### Treatment of Acute-Mucositis Induced Atrophy of the Mucosal Lining

The mucous membrane atrophy accompanying the primary acute oral mucositis is considered to be irreversible by the clinicians, in itself being disabling and also predisposing for further inflammation and/or infection events throughout the lifetime of the patient. However, the mucosal lining tissue macrophages can still be activated and as such it may be contemplated that the local host defense may be upregulated by local use of GM-CSF.

In order to avoid recurrent inflammation and/or infections potentially associated with an atrophic mucous membrane of the oral cavity, developed as a consequence of an acute oral mucositis, the present inventor has surprisingly found that administration of GM-CSF can regenerate the atrophic mucous membrane and upregulate the local host defense. In this way a normalized normal mucous membrane is obtained. In this way the risk of acquiring further inflammation and/or infection events are reduced or abolished. This is contrary to what the present inventor was informed by health care personnel, which informed that the atrophy caused by chemo-therapy-induced acute oral mucositis was generally irreversible, and further that local irritant drinks like alcoholic drink like wine, hypotonic beverages like water and spicy food most likely be excluded from oral intake due to pain-from oral intake due to pain forever.

GM-CSF is a relatively large protein, and water-soluble. Thus, it does not readily penetrate normal tissue structures. In order to regenerate the atrophic mucous membrane of the oral cavity, it is required that the GM-CSF can enter the target cells, which is possible when inflammation is on-going thus allowing GM-CSF to penetrate the mucous membrane. This is possible during an occurrence of post-therapeutic oral mucositis.

### Irradiation

In one embodiment, there is provided a composition comprising granulocyte-macrophage colony-stimulating factor (GM-CSF), or a functional variant or homologue thereof, for use in the treatment, prevention or alleviation of post-therapeutic oral mucositis, wherein said post-therapeutic oral mucositis is developed subsequent to a primary acute oral mucositis caused by radiation-therapy.

In one embodiment, said primary acute oral mucositis caused by radiation-therapy has subsided completely or partially and developed into a symptom-free stage characterized by atrophy of the mucosal lining of the oral cavity.

Radiation therapy, radiation oncology, therapeutic radiation or radiotherapy is the medical use of ionizing radiation, generally as part of cancer treatment to control or kill malignant cells. Radiation therapy may be curative in a number of types of cancer if they are localized to one area of the body. It may also be used as part of curative therapy, to prevent tumor recurrence after surgery to remove a primary malignant tumor. Radiation therapy is synergistic with chemotherapy, and has been used before, during, and after chemotherapy in susceptible cancers.

Radiation therapy is commonly applied to the cancerous tumor because of its ability to control cell growth. Ionizing radiation works by damaging the DNA of exposed tissue leading to cellular death. To spare normal tissues shaped radiation beams are aimed from several angles of exposure to intersect at the tumor, providing a much larger absorbed dose there than in the surrounding, healthy tissue. Besides the tumour itself, the radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion. The precise treatment intent (curative, adjuvant, neoadjuvant, therapeutic, or palliative) will depend on the tumor type, location, and stage, as well as the general health of the patient.

The amount of radiation used in photon radiation therapy is measured in gray (Gy), and varies depending on the type and stage of cancer being treated. For curative cases, the typical dose for a solid epithelial tumor ranges from 60 to 80 Gy, while lymphomas are treated with 20 to 40 Gy. Preventative (adjuvant) doses are typically around 45-60 Gy in 1.8-2 Gy fractions (for breast & head and neck cancers).

Radiation therapy also has several applications in non-malignant conditions, such as the treatment of trigeminal neuralgia, acoustic neuromas, severe thyroid eye disease, pterygium, pigmented villonodular synovitis, and prevention of keloid scar growth, vascular restenosis, and heterotopic ossification. The use of radiation therapy in non-malignant conditions is limited partly by worries about the risk of radiation-induced cancers.

For the purposes of the present invention, especially radiation therapy targeted at cancerous tissues of the head and/or neck area of a patient may cause mucositis. Targeting such cancers specifically with impose radiation on the oral cavity and induce changes in its mucosal lining, thereby potentially causing atrophy of the oral cavity, which is disabling per se and also predisposes for inflammation and/or infection.

### Chemotherapy

In one embodiment, there is provided a composition comprising granulocyte-macrophage colony-stimulating factor (GM-CSF), or a functional variant or homologue thereof, for use in the treatment, prevention or alleviation of post-therapeutic oral mucositis, wherein said post-therapeutic oral mucositis is developed subsequent to a primary acute oral mucositis caused by chemotherapy.

In one embodiment, said primary acute oral mucositis caused by chemotherapy has subsided and developed into a symptom-free stage characterized by atrophy of the mucosal lining of the oral cavity.

Chemotherapy is the treatment of cancer with an antineoplastic drug or with a combination of such drugs into a standardized treatment regimen. Certain chemotherapy agents also have a role in the treatment of other conditions, including ankylosing spondylitis, multiple sclerosis, Crohn's disease, psoriasis, psoriatic arthritis, rheumatoid arthritis, and scleroderma. The most common chemotherapy agents act by killing cells that divide rapidly, one of the main properties of most cancer cells. This means that chemotherapy also harms cells that divide rapidly under normal circumstances: cells in the bone marrow, digestive tract, and hair follicles.

Chemotherapeutic compounds according to the present invention may be any one of alkylating agents, anti-metabolites, plant alkaloids, terpenoids, topoisomerase inhibitors (type I and II), and cytotoxic antibiotics.

Systemically administered chemotherapeutics are known to enter the oral mucosa and potentially cause local damages to the oral cavity such as atrophy and acute oral mucositis.

Infection and/or Inflammation

Oral mucositis and the atrophy accompanying the symptom-free stage after the acute oral mucositis has subsided completely or partially, predisposes for subsequent inflammation and infection of the mucosal lining of the oral cavity. Associated with atrophy and ulceration of the oral mucosa is an increased risk of infection. The most common pathogenic agent is candida.

GM-CSF is known to enhance the host defense of a subject and may be used to treat infections (see e.g. WO 2008/052567). Thus, in an embodiment of the present invention there is provided GM-CSF, or variants or homologues thereof, to locally enhance the host defence of a subject in need thereof, such as the host defence of the oral cavity, thus reducing the risk of acquiring a local infection.

Inflammation is part of the complex biological response of vascular tissues to harmful stimuli, such as pathogens, damaged cells, or irritants. The classical signs of acute inflammation are pain (dolor), heat (calor), redness (rubor), swelling (tumor), and loss of function (functio laesa). Inflammation is a protective attempt by the organism to remove the injurious stimuli and to initiate the healing process. Inflammation is not a synonym for infection, even in cases where inflammation is caused by infection.

Infection is the invasion of a host organism's bodily tissues by disease-causing organisms, their multiplication, and the reaction of host tissues to these organisms and the toxins they produce. Infections are caused by microorganisms such as viruses, prions, bacteria, and viroids, and larger organisms like macroparasites and fungi. Hosts can fight infections using their immune system. Mammalian hosts react to infections with an innate response, often involving inflammation, followed by an adaptive response. Infections may for example be an infection by bacteria, fungi, viruses, parasites.

Factors which Promote Mucositis

Factors which can increase the likelihood of developing mucositis, or which can make it worse if it does occur, include: Diseases such as kidney disease, diabetes or HIV/AIDS, previous cancer treatment, chronic irritation from ill-fitting prostheses or faulty restorations can predispose patients to the development of oral mucositis due to local irritation and trauma.

In one embodiment, the compositions of the invention are for use in prophylactic or preemptive treatment, or in the treatment of oral mucositis which occur in connection with kidney disease.

In one embodiment, the compositions of the invention are for use in prophylactic or preemptive treatment, or in the treatment of oral mucositis which occur in connection with diabetes.

In one embodiment, the compositions of the invention are for use in prophylactic or preemptive treatment, or in the treatment of oral mucositis which occur in connection with HIV infection.

In one embodiment, the compositions of the invention are for use in prophylactic or preemptive treatment, or in the treatment of oral mucositis which occur in connection with AIDS.

In one embodiment, the compositions of the invention are for use in prophylactic or preemptive treatment, or in the treatment of oral mucositis which occur in connection with previous cancer treatment, such as chemotherapy or radiation therapy.

In one embodiment, the compositions of the invention are for use in prophylactic or preemptive treatment, or in the treatment of oral mucositis which occur in connection with chronic irritation from prostheses or faulty restorations.

Generally, patients with hematologic malignancies have an increased rate of oral mucositis compared with those with solid tumors. This is to some extent related to the treatment regimens.

In one embodiment, the compositions of the invention are for use in prophylactic or preemptive treatment, or in the treatment of oral mucositis which occur in connection with hematologic malignancies, such as in connection with or post treatment for the cancer.

Hyposalivation prior to and during treatment is associated with an increased risk of oral mucositis. In one embodiment, the compositions of the invention are for use in the preemptive treatment, prophylactic treatment or in the treatment of oral mucositis which occur in connection with hyposalivation. The condition of hyposalivation may according to the invention be in patients receiving chemotherapy or radiotherapy or both and/or other cancer therapy.

The use of methotrexate for chronic Graft-versus-host disease (GVHD) prophylaxis may exacerbate lesions of oral mucositis, although this is less of a concern with newer prophylaxis regimens. In one embodiment, the compositions of the invention are for use in the preemptive treatment, prophylactic treatment or in the treatment of oral mucositis which occur in connection with treatment with methotrexate, or other treatment, prophylaxis or preemptive treatment of GVHD.

Oral mucositis occurs independently of oral mucosal infections of viral and fungal etiology, but it may be exacerbated by such concomitant infections. In one embodiment, the compositions of the invention are for use in the treatment, preemptive treatment, or prophylactic treatment of mucositis which occur with concomitant infections of viral or fungal etiology.

In one embodiment, the compositions of the invention are for use in the treatment of oral mucositis which occur in connection with previous cancer treatment, such as chemotherapy or radiation therapy, and wherein the patient additionally suffer from any one of a kidney disease, diabetes or HIV/AIDS, chronic irritation from ill-fitting prostheses or faulty restorations, haematologic cancer, hyposalivation, GVHD, receive treatment with methotrexate, or where the patient has a concomitant infection of viral or fungal etiology.

### GM-CSF

Colony-stimulating factors (CSF) are glycoproteins that stimulate the growth of hematopoietic progenitors and enhance the functional activity of mature effector cells. In brief, at the level of immature cells, CSF's assure the self-renewal of the staminal pool and activate the first stage of hematopoietic differentiation; in the middle stage, when cell proliferation is associated to a progressive acquisition of characteristics of mature cells, they enormously enhance the number of differentiating cells; in the terminal stage they control the circulation and the activation of mature cells.

The cytokine according to the present invention is GM-CSF. Mature GM-CSF is a monomeric protein of 127 amino acids with several potential glycosylation sites. The variable degree of glycosylation results in a molecular weight range between 14 kDa and 35 kDa. Non-glycosylated and glycosylated GM-CSF show similar activity in vitro. There are two known sequence variants of GM-CSF. The active form of the GM-CSF protein is found extracellularly as a homodimer in vivo.

GM-CSF exerts its biological activity by binding to its receptor. The most important sites of GM-CSF receptor (GM-CSF-R) expression are on the cell surface of myeloid cells, like alveolar macrophages type I & II, epithelial pulmonary cells and endothelial cells, whereas lymphocytes are GM-CSF-R negative. The native receptor is composed of at least two subunits, alpha and beta. The alpha subunit imparts ligand specificity and binds GM-CSF with nanomolar affinity (Gearing et al., 1989; Gasson et al., 1986). The beta subunit is also part of the interleukin-3 and interleukin-5 receptor complexes and, in association with the GM-CSF receptor alpha subunit and GM-CSF, leads to the formation of a complex with picomolar binding affinity (Hayashida et al., 1990). The binding domains on GM-CSF for the receptor have been mapped: GM-CSF interacts with the beta subunit of its receptor via a very restricted region in the first alpha helix of GM-CSF (Shanafelt et al., 1991b & 1991a; Lopez et al., 1991). Binding to the alpha subunit could be mapped to the third alpha helix, helix C, the initial residues of the loop joining helices C and D, and to the carboxyterminal tail of GM-CSF (Brown et al., 1994).

Formation of the GM-CSF trimeric receptor complex leads to the activation of complex signaling cascades involving molecules of the JAK/STAT families, She, Ras, Raf, the MAP kinases, phosphatidylinositol-3-kinase and NFkB, finally leading to transcription of c-myc, c-fos and c-jun. Activation is mainly induced by the beta subunit of the receptor (Hayashida et al., 1990; Kitamura et al., 1991; Sato et al., 1993). The shared beta subunit is also responsible for the overlapping functions exerted by IL-3, IL-5 and GM-CSF (for review see: de Groot et al., 1998).

Apart from its hemopoietic growth and differentiation stimulating activity, GM-CSF functions especially as a proinflammatory cytokine. Macrophages, e.g. alveolar macrophages type I & II and monocytes as well as neutrophils and eosinophils become activated by GM-CSF, resulting in the release of other cytokines and chemokines, matrix degrading proteases, increased HLA expression and increased expression of cell adhesion molecules or receptors for CC-chemokinesm which in turn leads to increased chemotaxis of inflammatory cells into inflamed tissue.

Wong et al., Science Vol. 228, pp. 810-815 (1985) and Kaushansky et al., Proc. Natl. Acad. Sci. USA, Vol. 83, pp. 3101-3105 (1986) have described the production of recombinant GM-CSF in mammalian cells. Burgess et al., Blood, Vol. 69, pp. 43-51 (1987) describes the purification of GM-CSF produced in Escherichia coli.

In one embodiment, GM-CSF according to the present invention is recombinant GM-CSF (rGM-CSF). GM-CSF according to the present invention may be commercially available, e.g. sargramostim (GM-CSF [Leukine®; Immunex, Seattle, Wash.]).

The protein sequence of GM-CSF of Homo Sapiens (SEQ ID NO:1): MWLQSLLLLG TVACSISAPA RSPSPSTQPW EHVNAIQEAR RLLNLSRDTA AEMNETVEVI SEMFDLQEPT CLQTRLELYK QGLRGSLTKL KGPLTMMASH YKQHCPPTPE TSCATQIITF ESFKENLKDF LLVIPFDCWE PVQE

### Functional Homologues of GM-CSF

A functional homologue of GM-CSF is a polypeptide having at least 50% sequence identity with the known and naturally occurring sequence of GM-CSF and has one or more GM-CSF functions, such as the stimulation of the growth and differentiation of hematopoietic precursor cells from various lineages, including granulocytes, macrophages, eosinophils and erythrocytes.

GM-CSF regulates multiple functions of alveolar macrophages (AM). GM-CSF stimulation of AM has been documented to enhance alveolar macrophages selectively respond to noxious ingestants, i.e., stimulation of inflammation during bacterial phagocytosis, nonnoxious ingestants are generally mollified, i.e., antiinflammatory responses during phagocytosis of apoptotic cells. Further AM functions are enhanced by GM-CSF stimulation with subsequent proliferation, differentiation, accumulation and activation. Further these GM-CSF effects also encompasses cell adhesion, improved chemotaxis, Fc-receptor expression, complement- and antibody-mediated phagocytosis, oxidative metabolism, intracellular killing of bacteria, fungi, protozoa, and viruses, cytokine signaling, and antigen presentation. Further GM-CSF enhances defects in AM cell adhesion, pathogen associated molecular pattern receptors, like Toll-like receptors and TLR trans-membranous signaling, surfactant protein and lipid uptake and degradation (Trapnell B C and Whitsett J A. GM-CSF regulates pulmonary surfactant homeostasis and alveolar macrophage-mediated innate host defense. Annu. Rev. Physiol. 2002.64:775-802).

Further GM-CSF interacts with the AM's recognition receptors, the so-called toll like receptors (TLR). GM-CSF is important in the pulmonary host defense in pneumonia due to its interaction with the TLR's participation in the host defense resulting in enhanced clearance of the causative microorganism (Chen G H, Olszewski M A, McDonald R A, Wells J C, Paine R 3rd, Huffnagle G B, Toews G B. Role of granulocyte macrophage colony-stimulating factor in host defense against pulmonary Cryptococcus neoformans infection during murine allergic bronchopulmonary mycosis. Am J Pathol. 2007 March; 170(3):1028-40). Lung has its own innate GM-CSF production, which is reduced in pneumonia and hyperoxia, in relation to high O₂ exposure as seen in, e.g. ventilator associated pneumonia (VAP) contributing impairment of host defense secondary to apoptosis with poor response to infections. The hyperoxic injury seems to be counteracted by activation of alveolar macrophages with GM-CSF (Altemeier W A, Sinclair S E. Hyperoxia in the intensive care unit: why more is not always better. Curr Opin Crit Care. 2007 February; 13(1):73-8. & Baleeiro C E, Christensen P J, Morris S B, Mendez M P, Wilcoxen S E, Paine R.GM-CSF and the impaired pulmonary innate immune response following hyperoxic stress. Am J Physiol Lung Cell Mol Physiol. 2006 December; 291(6):L1246-55. Epub 2006 Aug. 4) with subsequent clearance of P. aeruginosa via expression of the TLR signaling pathway (Baleeiro C E, Christensen P J, Morris S B, Mendez M P, Wilcoxen S E, Paine R.GM-CSF and the impaired pulmonary innate immune response following hyperoxic stress. Am J Physiol Lung Cell Mol Physiol. 2006 December; 291(6):L1246-55. Epub 2006 Aug. 4).

Finally GM-CSF produces in-vitro conversion of AM into immature dendritic cells (DC), which may further be matured with specific agents in respect to activate the homing of matured DC's to a specified receptor or target. (Zobywalski A, Javorovic M, Frankenberger B, Pohla H, Kremmer E, Bigalke I, Schendel D J. Generation of clinical grade dendritic cells with capacity to produce biologically active IL-12p70. J Transl Med. 2007 Apr. 12; 5:18).

Evolutionary conservation between GM-CSF of different closely related species, e.g. assessed by sequence alignment, can be used to pinpoint the degree of evolutionary pressure on individual residues. Preferably, GM-CSF sequences are compared between species where GM-CSF function is conserved, for example but not limited to mammals including rodents, monkeys and apes. Residues under high selective pressure are more likely to represent essential amino acids that cannot easily be substituted than residues that change between species. It is evident from the above that a reasonable number of modifications or alterations of the human GM-CSF sequence does not interfere with the activity of the GM-CSF molecule according to the invention. Such GM-CSF molecules are herein referred to as functional equivalents of human GM-CSF, and may be such as variants and fragments of native human GM-CSF as described here below.

As used herein the expression “variant” refers to polypeptides or proteins which are homologous to the basic protein, which is suitably human GM-CSF, but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics. Within the meaning of the term “conservative amino acid substitution” as applied herein, one amino acid may be substituted for another within groups of amino acids characterised by having
i) polar side chains (Asp, Glu, Lys, Arg, His, Asn, Gln, Ser, Thr, Tyr, and Cys,)
ii) non-polar side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and/or
xi) amino acids being monoamino-dicarboxylic acids or monoamino-monocarboxylic-monoamidocarboxylic acids (Asp, Glu, Asn, Gln).

A functional homologue within the scope of the present invention is a polypeptide that exhibits at least 50% sequence identity with human GM-CSF, such as at least 60% sequence identity, for example at least 70% sequence identity, such as at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human GM-CSF, such as SEQ ID NO:1.

Sequence identity can be calculated using a number of well-known algorithms and applying a number of different gap penalties. Any sequence alignment algorithm, such as but not limited to FASTA, BLAST, or GETSEQ may be used for searching homologues and calculating sequence identity. Moreover, when appropriate any commonly known substitution matrix, such as but not limited to PAM, BLOSSUM or PSSM matrices, may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

Aside from the twenty standard amino acids and two special amino acids, selenocysteine and pyrrolysine, there are a vast number of “nonstandard amino acids” which are not incorporated into protein in vivo. Examples of nonstandard amino acids include the sulfur-containing taurine and the neurotransmitters GABA and dopamine. Other examples are lanthionine, 2-Aminoisobutyric acid, and dehydroalanine. Further non-standard amino are ornithine and citrulline.

Non-standard amino acids are usually formed through modifications to standard amino acids. For example, taurine can be formed by the decarboxylation of cysteine, while dopamine is synthesized from tyrosine and hydroxyproline is made by a posttranslational modification of proline (common in collagen). Examples of non-natural amino acids are those listed e.g. in 37 C.F.R. section 1.822(b)(4), all of which are incorporated herein by reference.

Both standard and non-standard amino acid residues described herein can be in the “D” or or “L” isomeric form.

It is contemplated that a functional equivalent according to the invention may comprise any amino acid including non-standard amino acids. In preferred embodiments a functional equivalent comprises only standard amino acids.

The standard and/or non-standard amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Suitably variants will be at least 60% identical, preferably at least 70% and accordingly, variants preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence of human GM-CSF.

Functional equivalents may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encom-passed within the present invention. Functional equivalents also comprise glycosylated and covalent or aggregative conjugates formed with the same molecules, including dimers or unrelated chemical moieties. Such functional equivalents are prepared by linkage of functionalities to groups which are found in fragment including at any one or both of the N- and C-termini, by means known in the art.

The term “fragment thereof” may refer to any portion of the given amino acid sequence. Fragments may comprise more than one portion from within the full-length protein, joined together. Suitable fragments may be deletion or addition mutants. The addition of at least one amino acid may be an addition of from preferably 2 to 250 amino acids, such as from 10 to 20 amino acids, for example from 20 to 30 amino acids, such as from 40 to 50 amino acids. Fragments may include small regions from the protein or combinations of these.

Suitable fragments may be deletion or addition mutants. The addition or deletion of at least one amino acid may be an addition or deletion of from preferably 2 to 250 amino acids, such as from 10 to 20 amino acids, for example from 20 to 30 amino acids, such as from 40 to 50 amino acids. The deletion and/or the addition may—independently of one another—be a deletion and/or an addition within a sequence and/or at the end of a sequence.

Deletion mutants of GM-CSF suitably comprise at least 20 or 40 consecutive amino acid and more preferably at least 80 or 100 consecutive amino acids in length. Accordingly such a fragment may be a shorter sequence of the sequence of human GM-CSF comprising at least 20 consecutive amino acids, for example at least 30 consecutive amino acids, such as at least 40 consecutive amino acids, for example at least 50 consecutive amino acids, such as at least 60 consecutive amino acids, for example at least 70 consecutive amino acids, such as at least 80 consecutive amino acids, for example at least 90 consecutive amino acids, such as at least 95 consecutive amino acids, such as at least 100 consecutive amino acids, such as at least 105 amino acids, for example at least 110 consecutive amino acids, such as at least 115 consecutive amino acids, for example at least 120 consecutive amino acids, wherein said deletion mutants preferably has at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human GM-CSF, such as SEQ ID NO:1.

The term “fragment thereof” may refer to any portion of the given amino acid sequence. Fragments may comprise more than one portion from within the full-length protein, joined together. Portions will suitably comprise at least 5 and preferably at least 10 consecutive amino acids from the basic sequence. They may include small regions from the protein or combinations of these.

There are two known variants of human GM-CSF; a T115I substitution in variant 1 and a I117T substitution in variant 2. Accordingly, in one embodiment of the invention functional homologues of GM-CSF comprises a sequence with high sequence identity to human GM-CSF NO: 1 or any of the splice variants.

Analogs of GM-CSF are for example described in U.S. Pat. Nos. 5,229,496, 5,393,870, and 5,391,485 to Deeley, et al. Such analogues are also functional equivalents comprised within the present invention.

In one embodiment GM-CSF is used according to the present invention in homo- or heteromeric form. Homo- and heteromeric forms of GM-CSF may comprise one or more GM-CSF monomers or functional homologous of GM-CSF as defined herein above. Homo- and heteromers include dimers, trimers, tetramers, pentamers, septamers, heptamers, octamers, nonamers and decamers. In one embodiment, a homodimer, trimer or tetramer of GM-CSF is used.

### Recombinant Production

Granulocyte-macrophage colony-stimulating factor (GM-CSF), or functional variants or homologues thereof, can be produced in various ways, such as isolation from for example human or animal serum or from expression in cells, such as prokaryotic cells, yeast cells, insect cells, mammalian cells or in cell-free systems.

In one embodiment of the invention, GM-CSF is produced recombinantly by host cells. Thus, in one aspect of the present invention, GM-CSF is produced by host cells comprising a first nucleic acid sequence encoding the GM-CSF operably associated with a second nucleic acid capable of directing expression in said host cells. The second nucleic acid sequence may thus comprise or even consist of a promoter that will direct the expression of protein of interest in said cells. A skilled person will be readily capable of identifying useful second nucleic acid sequence for use in a given host cell.

The process of producing a recombinant GM-CSF in general comprises the steps of: i) providing a host cell, ii) preparing a gene expression construct comprising a first nucleic acid encoding the GM-CSF operably linked to a second nucleic acid capable of directing expression of said protein of interest in the host cell, iii) transforming the host cell with the construct, and iv) cultivating the host cell, thereby obtaining expression of the GM-CSF, and optionally secretion of the GM-CSF into a culture medium.

The recombinant GM-CSF thus produced may be isolated by any conventional method, such as any of the methods for protein isolation described herein below. The skilled person will be able to identify a suitable protein isolation steps for purifying the GM-CSF. The composition comprising GM-CSF and nucleic acids may thus in this embodiment of the invention be the culture medium or a composition prepared from the culture medium. In another embodiment of the invention said composition is an extract prepared from animals, parts thereof or cells or an isolated fraction of such an extract.

In an embodiment of the invention, the GM-CSF is recombinantly produced in vitro in host cells and is isolated from cell lysate, cell extract or from tissue culture supernatant. In a more preferred embodiment the GM-CSF is produced by host cells that are modified in such a way that they express the relevant cytokine. In an even more preferred embodiment of the invention said host cells are transformed to produce and excrete the relevant GM-CSF.

### Pharmaceutical Composition

Pharmaceutical compositions or formulations for use in the present invention comprise GM-CSF, or functional variants or homologues thereof, preferably dissolved in a pharmaceutically acceptable carrier, preferably an aqueous carrier or diluent, or carried to the relevant site as a pegylated preparation or as a liposomal or nanoparticle preparation. A variety of aqueous carriers may be used, including, but not limited to saline, buffered saline, physiologically compatible buffers and the like.

Preferably, the composition comprises GM-CSF, or a variant or homologue thereof, in as aqueous suspension. Said suspension preferably comprises an adhesive. Said adhesive may in one embodiment be alginate (alginic acid), and in another embodiment methylcellulose. Adding an adhesive to the composition will increase the exposure time of the composition in the oral cavity and thus increase the efficacy of the composition.

The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration. In one embodiment a freeze-dried GM-CSF preparation may be pre-packaged for example in single dose units.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The formulations may contain pharmaceutically acceptable carriers and excipients including microspheres, liposomes, microcapsules, nanoparticles or the like. Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine] (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by e.g. Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multi-lamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some cases, it will be advantageous to include a compound, which promotes delivery of the active substance to its target.

Hyaluronic acid is a polymer of disaccharides themselves composed of D-glucoronic acids and D-N-acetylglucosamine.

Saccharides such as for example hyaluronic acid, sucrose hexa sulfate and sucrose octa sulfate have been associated with stimulation of wound healing and tissue repair. Saccharides may therefore have beneficial effect in combination with GM-CSF for treatment of post-therapeutic oral mucositis.

In one embodiment of the present invention, pharmaceutical compositions or formulations for use in the present invention comprise GM-CSF, or functional variants or homologues thereof, and further comprise one or more saccharides. Saccharides include nonsulfated saccharides such as for example hyaluronic acid, or sulfated saccharides having one or more sulfate groups, such as for example sucrose hexasulfate and/or sucrose octasulfate. Such saccharides may be formulated as a pharmaceutically acceptable salt. Of pharmaceutically acceptable salts, alkali metal salts such as potassium salt or sodium salts are preferred for the usage of the present invention.

Thus in a preferred embodiment, pharmaceutical compositions or formulations for use in the present invention comprise GM-CSF or functional variants or homologues thereof, and further comprise one or more saccharides, preferably hyaluronic acid and/or sucrose hexasulfate and/or sucrose octasulfate.

### Administration

An effective amount of GM-CSF, or functional variants or homologues thereof, according to the present invention are administered locally or orally. Thus, in a preferred embodiment, the composition comprising GM-CSF is administered directly to the oral cavity where its actions are required according to the present invention.

It one embodiment, a solution of GM-CSF, or functional variants or homologues thereof, according to the present invention, are administered directly to the oral cavity. In a preferred embodiment, a solution of GM-CSF, or functional variants or homologues thereof, is administered as a mouthwash or mouth rinse.

The mouthwash should preferably be contained in the mouth for a period, preferably by agitating or swirling of the solution within the oral cavity for a period of from 1 minute to 60 minutes; such as from 1-2 minutes, for example 2-3 minutes, such as from 3-4 minutes, for example 4-5 minutes, such as from 5-6 minutes, for example 6-7 minutes, such as from 7-8 minutes, for example 8-9 minutes, such as from 9-10 minutes, for example 10-15 minutes, such as from 15-20 minutes, for example 20-25 minutes, such as from 25-30 minutes, for example 30-40 minutes, such as from 40-50 minutes, for example 50-60 minutes. This will prolong the exposure time of the solution in the oral cavity.

Optimal administration of the mouthwash includes subsequent swallowing of the solution, thus targeting the entire oral cavity including also the epiglottis. Preferably, swallowing will occur by sinking part of the solution at a time thus prolonging exposure time of the solution at the epiglottis.

In one embodiment, the composition comprising GM-CSF according to the present invention is administered during an acute attack of post-therapeutic oral mucositis.

### Dose

By “effective amount” of GM-CSF according to the present invention it is meant a dose, which, when administered to a patient in need thereof, achieves a concentration which has a beneficial biological effect, i.e. by treating, alleviating and/or preventing post-therapeutic oral mucositis.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are normally of the order of several hundred μg active ingredient per administration with a preferred range of from about 0.1 μg to 10000 μg per dose. Doses expected to provide an effective amount of GM-CSF are often in the range of from 0.1 μg to 1 μg per dose, such as from 1 μg to 5 μg, for example 5 μg to 10 μg, such as from 10 μg to 25 μg, for example 25 μg to 50 μg, such as from 50 μg to 100 μg, for example 100 μg to 200 μg, such as from 200 μg to 300 μg, for example 300 μg to 400 μg, such as from 400 μg to 500 μg, for example 500 μg to 600 μg, such as from 600 μg to 750 μg, for example 750 μg to 1000 μg, such as from 1000 μg to 5000 μg, for example 5000 μg to 10000 μg per dosage. Preferably the dosage per administration is 100 to 1000 μg, such as 200 to 400 μg per dosage, preferably about 300 μg per dosage.

Each dose may be administered once a day, twice a day, three times a day, four times a day, five times a day or six times a day.

Duration of dosing will typically range from 1 day to about 4 months, such as in the range of 1 day to 2 days, for example 2 days to 3 days, such as in the range of 3 days to 4 days, for example 4-5 days, such as 5-6 days, for example 6-7 days, such as one week to two weeks, for example two to four weeks, such as one month to two months, for example 2 to 4 months, or as long as symptoms and disease is detectable. Preferably, duration occurs for around 14 days or more, in order to allow the patient to produce a fully immunocompetent dendritic cell from the resting macrophage (MF)—the so-called autocrine effect of the GM-CSF.

In some embodiments the solutions of the invention comprises an adhesive, a gelling agent or a thickener, to facilitate the adhesion to the mucous tissue in the oral cavity, throat and esophagus. This is to increase the exposure time of the solutions of the invention to the tissues. The adhesive, gelling agent or thickener may in non-limiting example be chosen from the list of alginate, hyaluronic acid or sucralfate or derivatives or variants of those.

Alginate may be present in the formulations of the present invention in an amount of between 5 and 500 mg/ml, such as between 5 and 250 mg/ml such as between 5 and 125 mg/ml, such as between 20 and 100 mg/ml. In one embodiment, alginate is present in about 50 mg/ml in the formulations of the present invention.

Hyaluronic acid may be present in the formulations of the present invention in an amount of between 5 and 500 mg/ml, such as between 5 and 250 mg/ml such as between 5 and 125 mg/ml, such as between 20 and 100 mg/ml. In one embodiment, alginate is present in about 50 mg/ml in the formulations of the present invention.

Sucralfate may be administered in a dosage ranging from 0.001 gram to 5 grams/ml, such as from 10 mg/ml to 1000 mg/ml such as from 10 mg/ml to 500 mg/ml, such as from 50 mg/ml to 500 mg/ml, such as about any one of 100 mg/ml, or 200 mg/ml or 250 mg/ml or 300 mg/ml or 400 mg/ml or 500 mg/ml.

In one preferred embodiment the composition of the invention comprises 300 microgram/ml GM-CSF, 10 wt/vol % sucralfate, and 5 wt/vol % hyaluronic acid. In one preferred embodiment the composition of the invention comprises from 100-500 microgram/ml GM-CSF, from 1-20 wt % sucralfate, and from 1-10 wt % hyaluronic acid. In one preferred embodiment the composition of the invention comprises from 200-400 microgram/ml GM-CSF, from 5-15 wt % sucralfate, and from 2.5-7.5 wt % hyaluronic acid.

### EMBODIMENTS

1. A composition comprising granulocyte-macrophage colony-stimulating factor (GM-CSF), or a functional variant or homologue thereof, for use in the treatment, prevention or alleviation of post-therapeutic oral mucositis.
2. The composition according to embodiment 1, wherein said post-therapeutic oral mucositis is associated with mucous membrane atrophy of the oral cavity.
3. The composition for use according to embodiment 2, wherein said atrophic mucous membrane of the oral cavity is regenerated.
4. The composition for use according to any of the preceding embodiments, wherein said post-therapeutic mucositis is subsequent to an acute mucositis caused by chemotherapy and/or radiotherapy, wherein said acute mucositis caused by chemotherapy/or radiotherapy has subsided partially or completely.
5. The composition for use according to any of the preceding embodiments, wherein said post-therapeutic mucositis is subsequent to a symptom-free stage characterized by atrophy of the oral mucosal lining, said symptom-free stage following an acute mucositis caused by chemotherapy and/or radiotherapy.
6. The composition for use according to any of the preceding embodiments, wherein said GM-CSF, or a functional variant or homologue thereof, is to be administered prior to and/or during and/or after chemotherapy and/or radiotherapy.
7. The composition for use according to any of the preceding embodiments, wherein said composition comprising GM-CSF, or a functional variant or homologue thereof, is an aqueous solution.
8. The composition for use according to embodiment 7, wherein said aqueous solution is to be administered orally.
9. The composition for use according to any of embodiments 7-8, wherein said aqueous solution to be administered orally is a mouth wash.
10. The composition for use according to any of embodiments 7-9, wherein said aqueous solution further comprises an adhesive, such as an alginate.
11. The composition for use according to any of the preceding embodiments, wherein said GM-CSF, or a functional variant or homologue thereof, is to be administered at an effective amount, such as from between 100 to 1000 microgram per dose, for example 200-800 microgram per dose, such as at about 300 microgram per dose.
12. The composition for use according to embodiment 11, wherein said dose is to be administered between one and ten times per day, such as between two and five times daily, for example once, twice, three times, four times or five times daily.
13. The composition for use according to embodiment 12, wherein said one or more daily doses are administered for between 7 days to several months, such as 14 days to 1 month.
14. The composition for use according to any of embodiments 8-13, wherein said aqueous solution is contained in the oral cavity by agitating or swirling of the solution within the oral cavity for a period of from 1 minute to 60 minutes; such as from 1-2 minutes, for example 2-3 minutes, such as from 3-4 minutes, for example 4-5 minutes, such as from 5-6 minutes, for example 6-7 minutes, such as from 7-8 minutes, for example 8-9 minutes, such as from 9-10 minutes, for example 10-15 minutes, such as from 15-20 minutes, for example 20-25 minutes, such as from 25-30 minutes, for example 30-40 minutes, such as from 40-50 minutes, for example 50-60 minutes.
15. A method for treating, preventing, reducing risk of, or alleviating of post-therapeutic oral mucositis in a subject in need thereof, said method comprising administering to the subject an effective amount of a composition comprising granulocyte-macrophage colony-stimulating factor (GM-CSF), or a functional variant or homologue thereof, as defined in any of claims 1 to 14.
   In one embodiment, the compositions of the invention are made for use in combination with other treatments, such as in non-limiting example any one of antibacterial, antiviral, antifungal, anticancer, or other treatment for mucositis or for treatment of some of the disorders which occur concomitant with the mucositis.

### Example I

A 68 year old man was diagnosed with tonsil cancer and underwent treatment therefore. Firstly the patient underwent extensive surgery with removal of the primary left-sided tumor and 5 metastases located in the pharynx and at the base of the tongue. Further 7 metastases located at the neck were also removed. Due to a remaining tumor residue cuffing around the carotis bifurcature, out of reach for surgical removal, the treatment was finalized with 32 sessions of radiation therapy. The radiation fields were addressing the right side of the neck, pharynynx, the primary tumor sites and the anatomical sites where the 12 metastases were located. The radiation therapy was supplemented with IV infusion with the chemotherapeutic drug cisplatin, based on the assumption that the tumor would exhibit increased sensitivity to the radiation intervention.

The patient was discharged after 3 months at which time he was declared free of disease.

Eight months after radiation the oral mucosa was typically clinically moderately hypotrophic mostly located on the right side where the radiation field was applied. During this period the most prominent signs and symptoms, i.e. the resting pain had subsided. The clinical picture became dominated by chronic dyphagia and severe dysarthria, combined with chronic pain and coughing, which was severely accentuated by inadvertent aspiration constantly related to every meal. The patient was told by the clinician that the complaints were due to sequelae after the radiation intervention, and as such after almost 1 year was now to be considered irreversible.

Nine months after the discharge the patient had a chlamydia pneumonia as a sign of decreaed host defense. All the signs and symptoms subsided after three days of macrolide therapy due to a chlamydia pneumonia infection, fully responsive to the treatment of a 500 mg macrolide daily for three days.

After another 3 weeks the symptoms of mucositis flared considerably up. The reason was a severe fungus Candida albicans infection, which subsided after four days treatment with oral Brentan ointment three times per day and oral Fluconazol 400 mg per day. The latter gave rise to a severe dysfunction with GI signs and symptoms. After four days intervention the patient had lost 5 kg body weight due to nausea and dysfunction of the swallowing function, the Candida infection defervesced. After the three days the appetite was normalized. After the fungal infection the oral mucositis signs and symptoms were severely aggravated.

The patient where then treated with an oral suspension of GM-CSF. The mouth and the pharynx were washed with at solution of 300 microgram GM-CSF dissolved/suspended in algenate (50 mg algenate per ml). The flask which contains total 50 ml mixture was washed into the oropharynx and kept there for some time about one minute and subsequently swallowed in small gulps in an amount of 3-4 ml per gulp. The procedure was repeated for 5-6 times per day.

The treatment lasted 10 days.

Already after 6-8 days the pain disappeared

Already after 6-8 days, the spontaneous soreness and pain in the mouth or throat was decreased, i.e. without aggravating factors as food ingestion. After another 2-3 weeks the spontaneous coughing when lying down at night was almost gone. After 6 weeks the severity of mucositis according to the WHO grading was reduced from grade 3 to 2. At this time the BWT was normalized.

However, the last sign and symptom to disappear, was the pain and coughing provoked by drinking and eating, decreased after 2 month, at which time the mucositis was decreased to a grade 1-2.

Interestingly the patient subsequently underwent surgery for inguinal hernia—an intervention that required endotracheal intubation. After the surgery, the anaesthetist in charge of the endotracheal intubation replied when asked directly on mucous membranes state —“they are completely normal—no sign of atrophy or even vulnerability with signs of increased mucosal vulnerability like bleeding.” After the extubation the patient had no cough or pain or bleeding. The patient subsequently switched to a new dentist: When asked whether she observed atrophy of the mucous membranes she stated: “Completely normal conditions without any redness or coatings”.

### CONCLUSION AND FINAL REMARKS

Firstly the local host defence stayed normal without any blurr up or increase in mucositis grade throughout the next period of another year.

Secondly the present case also show that the said atrophy was not irreversible as postulated by the onchologist. The reason was that the clinic never used oral GM-CSF.

It has been documented (Rose et al.) that the GM-CSF does not spill-over from air to blood through intact biological membranes like the alveolo-capillary membrane. Considering the early improvement—it could be inferred that there must have been a certain passage of the drug through the inflamed membrane.

1. A composition comprising:
a granulocyte-macrophage colony-stimulating factor (GM-CSF), or a functional variant or homologue thereof, for use in the treatment, prevention or alleviation of oral mucositis.

2-18. (canceled)


SEQUENCE LISTING (1 sequence)

SEQ ID NO: 1 - protein, 144 aa, Homo Sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE